(19) United States
(12) Patent Application Publication
(10) Pub. No.: US 2023/0172458 A1
(43) Pub. Date: Jun. 8, 2023

(54) SMART MACHINES AND MACHINE LEARNING FOR HEMODYNAMIC SUPPORT DEVICES

(71) Applicant: ABIOMED, Inc., Danvers, MA (US)

(72) Inventors: Christian Moyer, Danvers, MA (US); Elise Jortberg, Danvers, MA (US); Dawn Bardot, Danvers, MA (US); Govind Bhala, Danvers, MA (US); Maximilian Maier, Danvers, MA (US); Eric Chase, Danvers, MA (US); Christoph Griesshammer, Danvers, MA (US)

(73) Assignee: ABIOMED, Inc., Danvers, MA (US)

(21) Appl. No.: 18/073,067

(22) Filed: Dec. 1, 2022

Related U.S. Application Data
(60) Provisional application No. 63/285,302, filed on Dec. 2, 2021.

(51) Int. Cl. A61B 5/02; A61B 5/00

(52) U.S. Cl. CPC A61B 5/02028; A61B 5/7275; A61B 5/742; A61B 5/746; A61B 5/7267

(57) ABSTRACT

Methods and systems are provided that utilize smart hemodynamic support devices positioned in one area of the body, in combination with machine learning to infer and/or detect conditions in other areas of the body operably connected via blood flow.

### CROSS-REFERENCE TO RELATED APPLICATIONS

The present application claims priority to U.S. Provisional Pat. App. No. 63/285,302, filed Dec. 2, 2021, the entirety of which is incorporated by reference herein.

### TECHNICAL FIELD

The present disclosure is drawn to hemodynamic support devices, and specifically devices that infer problems in one area of the subject's body based on conditions detected in other areas of the body.

### BACKGROUND

Conventional medical devices, designed to be inserted into or onto specific locations of a patient's body, routinely incorporate sensors. Such sensors reflect the conditions of wherever the medical device has been positioned. While the information received from the sensors can be displayed, and ranges for normal operation determined, such approaches have numerous downsides. For example, when the information from a sensor is determined to fall outside a normal range, such approaches may not be able to determine if there is an actual problem and/or the most likely cause of the problem.

### BRIEF SUMMARY

In some embodiments, a system for detecting and/or inferring conditions may be provided. The system may include a hemodynamic support device configured to be positioned in a first area of a subject's body, and at least one processor operably coupled to the hemodynamic support device. The at least one processor may be configured to receive data from the hemodynamic support device. The at least one processor may also be configured to determine, with at least one trained machine learning (ML) algorithm, a first probability of a condition existing in a second area of the subject's body based on the received data, the second area being different from the first area.

In some embodiments, the at least one processor may include a first processor and a second processor. The first processor may be configured to receive data from the hemodynamic support device and transmit the data to a second processor over a network. The second processor may be configured to receive data from the first processor, and determine, with the trained machine learning algorithm, the first probability of the condition existing in the second area of the subject's body based on the received data. In some embodiments, the second processor may be further configured to transmit the first probability to the first processor.

In some embodiments, the system may include a remote device. In some embodiments, the second processor may be further configured to transmit the first probability to the remote device. The remote device may be configured to display the first probability, or a text or image representative of the first probability. In some embodiments, the remote device may be configured to send the second data and third data to the at least one processor, and may be configured to receive the first probability, second probability, and third probability from the at least one processor. In some embodiments, no user-identifiable data is transmitted to or from the remote device. In some embodiments, the remote device is a mobile phone, tablet, or laptop. A user, such as a nurse, clinician, medical personnel, etc., may be the user of the remote device.

In some embodiments, the at least one trained ML algorithm may include a first ML algorithm trained on historical data gathered from a plurality of medical device models (e.g., some or all of Abiomed's Impella® blood pumps models). In some embodiments, the at least one trained ML algorithm may include a second ML algorithm trained on data gathered from a single medical device model (e.g., only Abiomed's Impella® 5.5 with Smart Assist)

In some embodiments, the system may be configured to continue refining probability estimates after making the first probability determination, based on new information sent to the processor(s).

In some embodiments, the at least one processor may be further configured to receive second data (e.g., entered by a user on a remote device, or sent to the processor(s) from a medical device, etc.), after receiving the data from the hemodynamic support device. The second data may relate to a third area of the subject's body that is different from the first area and the second area. For example, if the first area is the patient's left heart, and the second area is the patient's right heart, the third area may be the patient's vena cava. In some embodiments, the second data may include a value relating to a central venous pressure (CVP). In some embodiments, the at least one trained ML algorithm may be further configured to determine a second probability of the condition based on the data from the hemodynamic support device and the second data.

In some embodiments, the at least one processor may be configured to receive third data, after receiving the second data, the third data relating to a fourth area of the subject's body that is different from the first area, second area, and third area. For example, if the first, second, and third areas are the left heart, right heart, and vena cava, the fourth area may be, e.g., the pulmonary artery. In some embodiments, the third data may include a value relating to pulmonary artery pulsatility (PAP). In some embodiments, the at least one trained ML algorithm may be further configured to determine a third probability of the condition based on the data from hemodynamic support device, second data, and third data.

The at least one trained ML algorithm may also be trained to consider derived data. In some embodiments, the at least one processor may be further configured to derive at least one parameter, and the at least one trained ML algorithm may be configured to determine a probability (such as the first, second, or third probability as disclosed herein) further based on the at least one parameter, where the at least one parameter may be central venous pressure (CVP), a right atrial pressure (RAP), a min, max, and/or mean of RAP, right ventricle end-diastolic pressure (RVEDP), pulmonary artery pressure (PAP), a mean, systolic, and/or diastolic PAP, a pulmonary artery pressure index (PAPI), and/or an echo based parameter of right heart function. In some embodiments, PAPI may be calculated by subtracting a diastolic pulmonary artery value (PAdia) from a systolic pulmonary artery value (PAsys), and the difference is then divided by RAP or CVP. In some embodiments the echo-based parameter of right heart function may be a right ventricle (RV) diameter, a RV volume, a RV stroke volume index (RVSVI) value, a RV stroke work index (RVSWI) value, and/or a tricuspid annular plane systolic excursion (TAPSE) value.

In some embodiments, the at least one processor may be further configured to derive at least one parameter, and the at least one trained ML algorithm may be configured to determine a probability (such as the first, second, or third probability as disclosed herein) further based on the at least one parameter, where the at least one parameter may be a LV end-diastolic pressure (LVEDP), a pump suction, a pump alarm rate and/or type, a cardiac power output, a LV contractility, a LV relaxation, a pulse wave velocity, an ejection fraction, a statistical metric of a parameter included in the data from the hemodynamic support device, and/or a systolic value, diastolic value, mean, median, min, max, delta, or pulse of a parameter included in the data from the hemodynamic support device.

In some embodiments, the system may include other data-gathering devices. For example, in one embodiment, the system may include an additional device (or devices) operably coupled to the at least one processor. The additional device(s) may include a sensor, where the sensor may be positioned in or on a third area of the patient's body. For example, a watch with a sensor may be placed around a subject's wrist, or a patch with a sensor may be placed on a subject's chest.

In some embodiments, the at least one trained ML algorithm may be configured to determine a probability (such as the first probability, second probability, or third probability as disclosed herein) further based on data received from the sensor of the additional device.

In some embodiments, the data received from the sensor of the additional device may include a heart rate, a value related to blood oxygen, a value relating to an electrocardiogram (ECG), a skin temperature, or an acceleration.

In some embodiments, the data from the hemodynamic support device may include first information related to left heart contractile function, and second information related to suction or pump flow in the left heart. In some embodiments, the first information may include a left ventricle (LV) contractility value, an aortic (AO) pulse pressure and/or pulsatility value, or a combination thereof. In some embodiments, the data from the hemodynamic support device may include aortic (AO) pressure, left ventricular (LV) pressure, pump motor speed, pump motor current, LV-AO pressure gradient, pump flow, cardiac output, native cardiac output, LV pulse rate, AO pulse rate, or a combination thereof. In some embodiments, the data from the hemodynamic support device may also include LV volume (e.g., via conductance), heart rate, heart rhythm, arterial pressure, blood oxygenation, or a combination thereof

In some embodiments, the at least one processor may be further configured to analyze the determined probability in various ways. In some embodiments, the processor(s) may be configured to determine if the first probability is above a first threshold and/or below a second threshold. In some embodiments, the processor(s) may be configured to determine a trend over time in probabilities of the condition in the subject. In some embodiments, the processor(s) may be configured to determine if a rate of change described by the trend is above a threshold rate and/or if the probability will be above the first threshold or below the second threshold within a predetermined period of time should the trend continue. In some embodiments, the processor(s) may be configured to identify one or more primary factors that result in the probability being above the first predetermined threshold and/or below the second predetermined threshold. In some embodiments, the processor(s) may be configured to determine a trend over time in identified primary factors. In some embodiments, various combinations of these are utilized.

In some embodiments, the at least one processor may be further configured to alert a user when the first probability is determined to be above the first predetermined threshold or below the second predetermined threshold, when the rate of change described by the trend is above the threshold rate, and/or when it is determined, should the trend continue, the probability will be above the first threshold or below the second threshold within the predetermined period of time.

In some embodiments, the at least one processor may be further configured to receive input from the user responsive to the alert. For example, the input responsive to the alert may include data indicating a particular treatment was undertaken, or an existing treatment was stopped. In some embodiments, the alert may include options for the user to select. In some embodiments, the options may include tests and/or treatments for the condition.

In some embodiments, the at least one processor may be further configured to track the probability of the condition over time to determine if a provided treatment is effective at lowering risk of the condition.

In some embodiments, a method for detecting and/or inferring conditions may be provided. The method may include receiving data from a hemodynamic support device positioned in a first area of a subject's body. In some embodiments, the data may include, e.g., aortic (AO) pressure, left ventricular (LV) pressure, pump motor speed, pump motor current, LV-AO pressure gradient, pump flow, cardiac output, native cardiac output, LV pulse rate, AO pulse rate, or a combination thereof. In some embodiments, the data may also include LV volume via conductance, heart rate, heart rhythm, arterial pressure, blood oxygenation, or a combination thereof.

The method may include determining, with a trained machine learning algorithm, a first probability of a condition existing in a second area of the subject's body based on the received data, the second area being different form the first area.

In some embodiments, the data may be received from the hemodynamic support device by a first device (such as a local controller with a first processor), the first probability may be determined by a second device (such as a remote server with a second processor). In some embodiments, the method may include transmitting the data over a network to the second device. In some embodiments, the method may include transmitting the first probability to the first device. In some embodiments, the method may include transmitting the first probability to a third device, the third device being configured to display the first probability, or a text or image representative of the first probability.

In some embodiments, the method may include providing a first ML algorithm trained on historical data gathered from a plurality of medical device models. In some embodiments, the method may include providing a second ML algorithm trained on data gathered from a single medical device model.

In some embodiments, the method may include receiving second data (e.g., from a user-controlled device, such as a mobile phone, or from a medical device controlled by the user), after receiving the data from the hemodynamic support device, the second data relating to a third area of the subject's body that is different from the first area and the second area. In some embodiments, the method may include determining, using the at least one trained ML algorithm, a second probability of the condition based on the data from the hemodynamic support device and the second data. In some embodiments, the method may include receiving third data (e.g., from the user-controlled device, etc.), after receiving the second data from the user, the third data relating to a fourth area of the subject's body that is different from the first area, second area, and third area. In some embodiments, the method may include determining, using the at least one trained ML algorithm, a third probability of the condition based on the data from hemodynamic support device, second data, and third data.

As disclosed herein, the method may also include deriving at least one parameter, which may be used to determine one or more probabilities. Such parameters may include central venous pressure (CVP), a right atrial pressure (RAP), a min, max, and/or mean of RAP, right ventricle end-diastolic pressure (RVEDP), pulmonary artery pressure (PAP), a mean, systolic, and/or diastolic PAP, a pulmonary artery pressure index (PAPI), an echo based parameter of right heart function, a LV end-diastolic pressure (LVEDP), a pump suction, a pump alarm rate and/or type, a cardiac power output, a LV contractility, a LV relaxation, a pulse wave velocity, an ejection fraction, a statistical metric of a parameter included in the data from the hemodynamic support device, and/or a systolic value, diastolic value, mean, median, min, max, delta, or pulse of a parameter included in the data from the hemodynamic support device, or a combination thereof.

In some embodiments, the method may include sending the first probability, second probability, and third probability to a user-controlled device (such as a mobile phone, tablet, laptop, etc.). The method may include preventing user-identifiable data from being transmitted to or from the user-controlled device.

In some embodiments, the method may include receiving data from an additional device (such as a watch or patch) including a sensor, the sensor being positioned in or on a third area of the patient's body. The data received from the additional device may include, e.g., a heart rate, a value related to blood oxygen, a value relating to an electrocardiogram (ECG), a skin temperature, or an acceleration. In some embodiments, the at least one trained ML algorithm may determine the first probability further based on data received from the sensor of the additional device.

In some embodiments, the method may include determining if the first probability is above a first threshold and/or below a second threshold. In some embodiments, the method may include determining a trend over time in probabilities of the condition in the subject, and optionally determining if a rate of change described by the trend is above a threshold rate and/or if the probability will be above the first threshold or below the second threshold within a predetermined period of time should the trend continue. In some embodiments, the method may include identifying one or more primary factors that result in the probability being above the first predetermined threshold and/or below the second predetermined threshold. In some embodiments, the method may include determining a trend over time in identified primary factors.

In some embodiments, the method may include alerting a user when certain conditions are met. In some embodiments, a user may be alerted when the first probability is determined to be above the first predetermined threshold or below the second predetermined threshold, when the rate of change described by the trend is above the threshold rate, and/or when it is determined, should the trend continue, the probability will be above the first threshold or below the second threshold within the predetermined period of time.

In some embodiments, the method may include receiving input from the user responsive to the alert. In some embodiments, the input responsive to the alert may include data indicating a particular treatment was undertaken, or an existing treatment was stopped. In some embodiments, the alert may include options for the user to select, the options including tests and/or treatments for the condition. In some embodiments, the method may include tracking the probability of the condition over time. In some embodiments, the method may include determining if a provided treatment is (or was) effective at lowering risk of the condition.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic illustration of an embodiment of a system according to one embodiment of the present disclosure.

FIG. 2 is a representation of an embodiment of a user interface.

FIGS. 3A-3C are block diagrams describing embodiments of machine learning models configurations.

FIG. 4 is a flowchart showing an embodiment of a validation and training process.

### DETAILED DESCRIPTION

The following description and drawings illustrate the principles of the present disclosure. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the disclosure and the concepts contributed by the inventors to furthering the art and are to be construed as being without limitation to such specifically recited examples and conditions. Additionally, the term, “or” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the teachings disclosed herein. In general, statements made in the specification of the present application do not necessarily limit any of the various claimed disclosure. Moreover, some statements may apply to some disclosed features but not to others.

Disclosed herein are methods and systems utilizing smart hemodynamic support devices positioned in one area of a subject's body and machine learning to infer and/or detect conditions in other areas of the subject's body, such as those operably connected via blood flow. For example, the inventors have appreciated the benefits of the disclosed methods and systems that may allow for the detection of right heart failure (RHF) from signals received from a pump that may be positioned in the left heart.

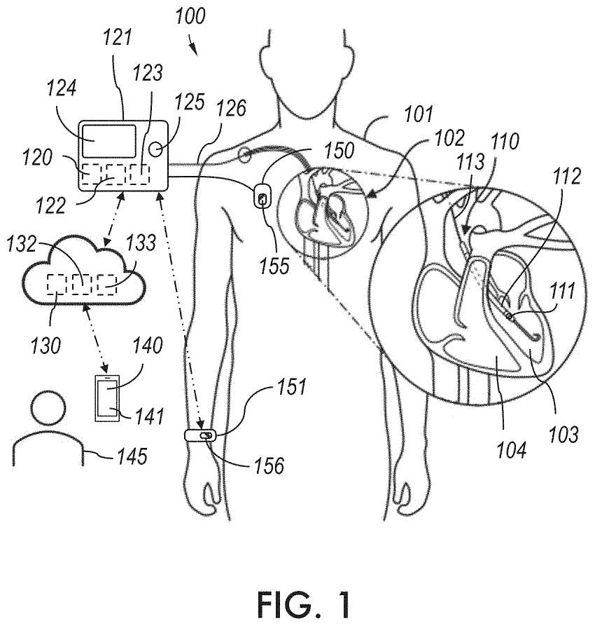

In some embodiments, a system for detecting and/or inferring conditions may be provided. Referring to FIG. 1, the system 100 may include a hemodynamic support device 110 configured to be positioned in a first area 103 of a body of a subject 101. The hemodynamic support device may include, e.g., a blood pump 112, which may be coupled to a distal end of a catheter 113. In FIG. 1, the blood pump is shown as being placed in the heart 102, and specifically in the left heart.

The system may include at least one processor, such as processor 120. The processor may be operably coupled to the hemodynamic support device, either via a wired or wireless connection. In some embodiments, the hemodynamic support device may be removably coupled via one or more wires 126 to a controller 121. The controller may include a processor 120 operably coupled to a memory 122, a non-transitory computer-readable storage medium 123, a display 124, and/or one or more controls 125, such as mechanical controls (e.g., buttons or knobs) or non-mechanical controls (e.g., touch screens). As will be understood, the non-transitory computer-readable storage medium may contain instructions that, when executed by the processor, configure the processor to perform certain steps.

The hemodynamic support device may include one or more sensors 111. Such sensors may be any appropriate sensor for the intended data to be collected, as described herein, and may include, e.g., electrodes, optical sensors and/or pressure sensors.

In some embodiments, the at least one processor may be configured to receive data from the hemodynamic support device.

In some embodiments, the data from the hemodynamic support device may include, e.g., an aortic (AO) pressure, left ventricular (LV) pressure, pump motor speed, pump motor current, LV-AO pressure gradient, pump flow, cardiac output, native cardiac output, LV pulse rate, AO pulse rate, or a combination thereof. In some embodiments, the data may also include LV volume (e.g., via conductance), heart rate, heart rhythm, arterial pressure, blood oxygenation, or a combination thereof

In some embodiments, the processor(s) may be configured to determine or derive a parameter based on the data received from the hemodynamic support device. In some embodiments, the at least one parameter may be: a LV end-diastolic pressure (LVEDP); a pump suction; a pump alarm rate and/or type; a cardiac power output (CPO); a LV contractility; a LV relaxation; a pulse wave velocity; an ejection fraction; a statistical metric of a parameter included in the data from the hemodynamic support device; and/or a systolic value, diastolic value, mean, median, minimum, maximum, delta, or pulse of a parameter included in the data from the hemodynamic support device.

In some embodiments, cardiac power output (CPO) may be calculated by mean arterial pressure (MAP)×CO (cardiac output)/451, where mean arterial pressure (MAP)=[(systolic blood pressure−diastolic blood pressure)/3]+diastolic blood pressure.

The one or more processors maybe configured to determine, with at least one trained machine learning (ML) algorithm, a first probability of a condition existing in a second area of the subject's body based on the received data, the second area being different from the first area.

In some embodiments, the condition may be right heart failure (RHF), but additional, or alternative, conditions also may be incorporated as well, based on where the hemodynamic support device is placed.

In some embodiments, the data from the hemodynamic support device may include first information related to functioning of the area in which the hemodynamic support device is placed, and second information related to functioning of the hemodynamic support device. In some embodiments, the first information may include information relating to left heart contractile function (for example, left ventricle (LV) contractility, aortic (AO) pulse pressure and/or pulsatility, or a combination thereof), and second information related to suction or pump flow in the left heart.

In some embodiments, the processor on the controller may include the at least one trained ML algorithm, and thus, may be configured to make such determinations locally. In some embodiments, the processor 120 may be configured to display some or all of the data received from the hemodynamic support device on the display 124. In some embodiments, the processor 120 may be configured to display some all of the determinations from the ML algorithm(s) (e.g., of one or more probabilities). These values may be displayed in any appropriate manner; for example, in some embodiments, a number or other text may be displayed, a graph or trend may be displayed, or both.

In some embodiments, the local controller may not be configured to make the determinations. In some embodiments, the at least one processor may include a first processor (such as processor 120) and a second processor (such as processor 130), which may be located remotely, such as in a cloud-based server, etc. As will be understood, the second processor, like the first, may be coupled to a memory 132 and a non-transitory computer-readable storage medium 133.

In some embodiments, the first processor may be configured to receive data from the hemodynamic support device and transmit the data to the second processor over a network.

In some embodiments, the second processor may be configured to receive data from the first processor, and to determine, with the trained machine learning algorithm, the first probability of the condition existing in a different area of the subject's body based on the received data. That is, the second processor may incorporate the one or more trained ML algorithms, rather than a local controller, for example.

In some embodiments, the second processor (e.g., processor 130) may be configured to transmit the determined probability to the first processor (e.g., processor 120). The first processor may then display the probabilities, or a representation of the probabilities, on the display.

In some embodiments, the system may include a remote device 140, which may be associated with a user 145. For example, the user may include a healthcare provider, a researcher, the subject, or an aide.

In some embodiments, the remote device may be a mobile phone, laptop, tablet, or other computer or computing device. The remote device may include a display 141, which may be a touch-sensitive display. The remote device may be configured to provide a user interface for the user, such as to allow the user to interact with the remote device. The user interface may include one or more screens to be displayed to the user. The remote device may include an input device (not shown), which may be include the touch-sensitive display, a mouse, a keyboard, etc., to allow the user to make a selection, to enter data, and/or to interact with the user interface.

In some embodiments, the processor(s) determining the probability (such as processor 120 or processor 130) may be further configured to transmit the first probability to the remote device. This may be transmitted over one or more networks, such as over the internet. In some embodiments, the remote device may be configured to display the first probability, or a text or image representative of the first probability.

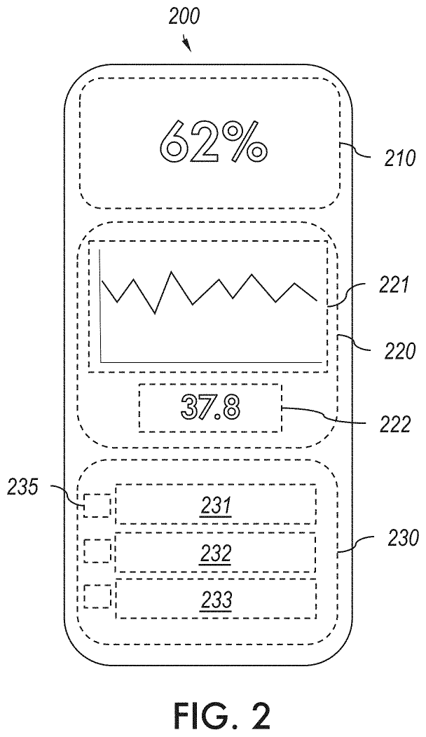

A user interface may be seen in FIG. 2, which may be seen on the remote device and/or the controller, for example. The user interface may include one or more display screens 200, each of which may be divided into one or more sections, such as a first section 210, a second section 220, and a third section 230. Each section may be configured to display different information.

In some embodiments, a first section may display the most recently determined probability in the first section 210. Here, that is shown as a specific value of “62%”, although it will be understood that a range of values (such as “>95%” or “70-80%”, etc.) may be displayed in some embodiments. In some embodiments, this display may be provided as a text or image representative of the first probability. For example, the interface may display text that is a description of the probability (such as, e.g., “low”, “medium”, “high” risk or probability of the condition, or a rating, such as a rating on a scale of 1-5, or terms defining ranges of probabilities, such as “unlikely”, “possible”, “likely”, “very likely”, etc.). These values or text representations may be color coded, such as green for probabilities below 50%, yellow for probabilities above 50% but below 75%, and red for probabilities above 75%. Alternatively, or in addition, the interface may display an image, which may be a rating, such as a star rating of 1-5 stars, a graph showing the probability data over time, an icon, such as up and down arrows, or a color-coded representation (e.g., red, yellow, or green tiles to indicate determined probability).

In some embodiments, the second section 220 may display additional data, which may include one or more graphs 221 and one or more text fields 222, for example, which may include words or numbers describing some or all of the data received, derived, or determined by the one or more processors. In some embodiments, this may include one or more graphs showing trends in data, or bar charts showing data broken down by pump speed setting, for example. In some embodiments, this may include one or more text fields displaying a description and/or a value (here, only “37.8” is displayed) for a parameter that may be of interest to the user.

In some embodiments, the third section 230 may include one or more text fields, which may include a text field 231 for describing a treatment option, a data entry field 232 for allowing a user to enter a requested value (such as a CVP value), and/or a field 233 for additional data for the user to enter information into such as a particular alternate treatment undertaken, for example. The third second also may include one or more selectable buttons 235 that may indicate the user has read a particular text field, allow a user to enter data into a field, submit the data entered into a field to the one more processor(s), and/or request assistance.

In some embodiments, if more information is provided than can appear on a single screen, the entire display may be scrolled up and down for proper viewing. In some embodiments, the information within each section may be scrolled independently.

In some embodiments, the system may include a graphical interface to display the signals and/or information determined from the signals received from the hemodynamic support device. As will be appreciated the graphical interface may be on one or more devices, including, e.g., a patient console, a computer, and/or a mobile device, etc. The graphical user interface may be the same or different on each display.

As will be appreciated, in some embodiments, the screen may be configurable by the user. For example, in some embodiments, the user may decide which information (e.g., which sections) may be visible on the screen (e.g., to avoid having to scroll up and down to view the desired information).

In some embodiments, the one or more trained ML algorithms may be based on historical data, which may include data gathered from devices other than hemodynamic support devices, and/or may include data gathered from one or more hemodynamic support device models. For example, in some embodiments, the training may occur using data gathered from some or all Impella® blood pumps offered by Abiomed Inc. In some embodiments, the training may occur using data from only a single hemodynamic support model; for example, from data gathered only from specific blood pump, such as the Impella® CP blood pumps offered by Abiomed Inc.

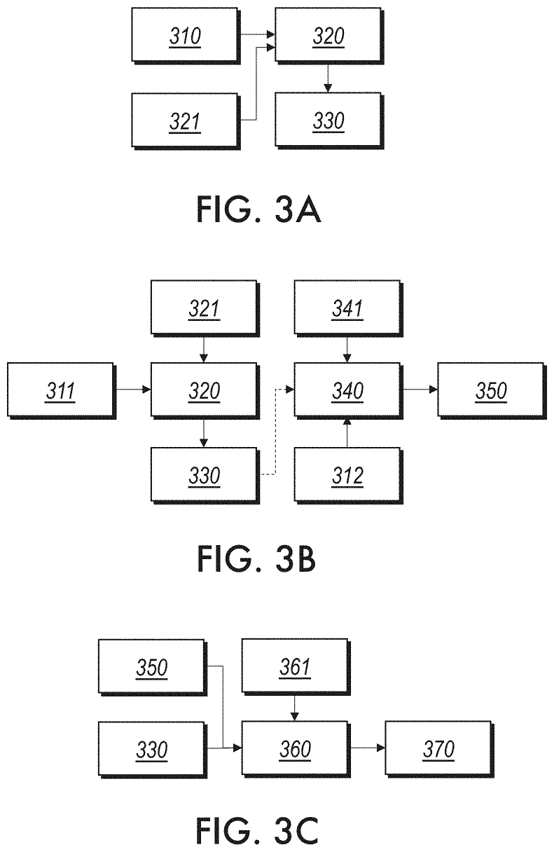

Referring to FIG. 3A, in some embodiments, the system may include a database 310 that includes all data used for training and validation purposes. Such data may be gathered from multiple databases, and may include, e.g., data such as results from Abiomed Inc.'s Global cVAD study, and data logs from blood pump controllers. A training dataset can be fed to a ML algorithm (running on a processor), along with various parameters 321 (e.g., measured or derived parameters, including but not limited to CVP, RAP, min, max, and/or mean of RAP, RVEDP, PAP, mean, systolic, and/or diastolic PAP, PAPI, an echo based parameter of right heart function, LVEDP, a pump suction, a pump alarm rate and/or type, a cardiac power output, a LV contractility, a LV relaxation, a pulse wave velocity, an ejection fraction, a statistical metric of a parameter included in the data from the hemodynamic support device, and/or a systolic value, diastolic value, mean, median, min, max, delta, or pulse of a parameter included in the data from the hemodynamic support device, or a combination thereof), resulting in a trained model 330, which can then be validated, and used for making the disclosed probabilities, etc., as understood in the art.

In some embodiments, rather than a single model being created from a large database, two or more models may be created. For example, in one embodiment, seen in FIG. 3B, a first database 311 is used to train a first model 330, and a second database 312 is used to train a second model 350. In such embodiments, the first database may include historical data from multiple sources, while the second database may include only the data relating to the specific model being introduced to the subject (e.g., if an Impella® CP blood pump is positioned in a subject's heart, the second model would be trained using data gathered only from Impella® CP blood pumps). Here, a training dataset from the first database can be fed to a ML algorithm (running on a processor), along with various parameters 321, resulting in a first trained model 330, which can then be validated, and used for making the disclosed probabilities, etc., as understood in the art. In some embodiments, data from the second dataset may be fed to a second ML algorithm (running on a processor), along with various parameter 341, resulting in a second trained model 350. In some embodiments, the first trained model may be fed to the second ML algorithm along with data from the second dataset and various parameters.

In some embodiments, these ML models may be configured to output a probability of a condition occurring. In some cases, each model may independently determine a probability of the condition occurring, and these independent determinations may be combined to determine a final probability. In some embodiments, a third ML algorithm may be trained my providing the trained models 330, 350 to a third ML algorithm (running on a processor), along with various parameters 361, resulting in a third trained model 370.

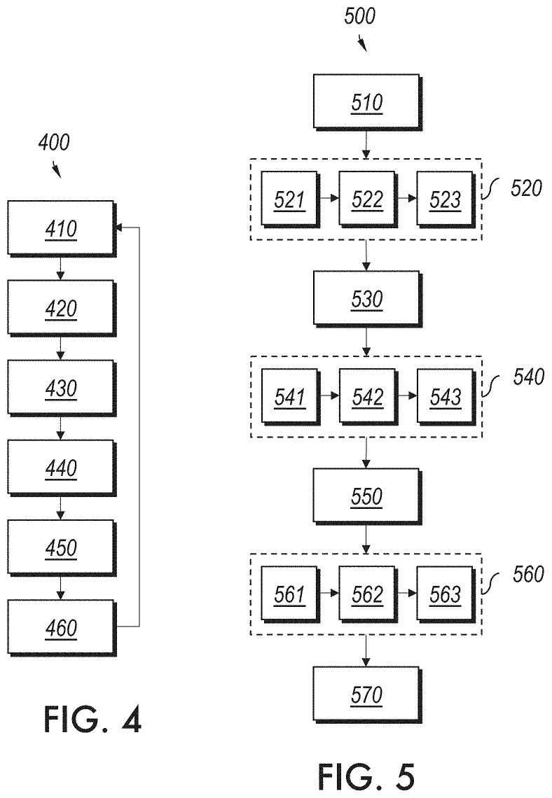

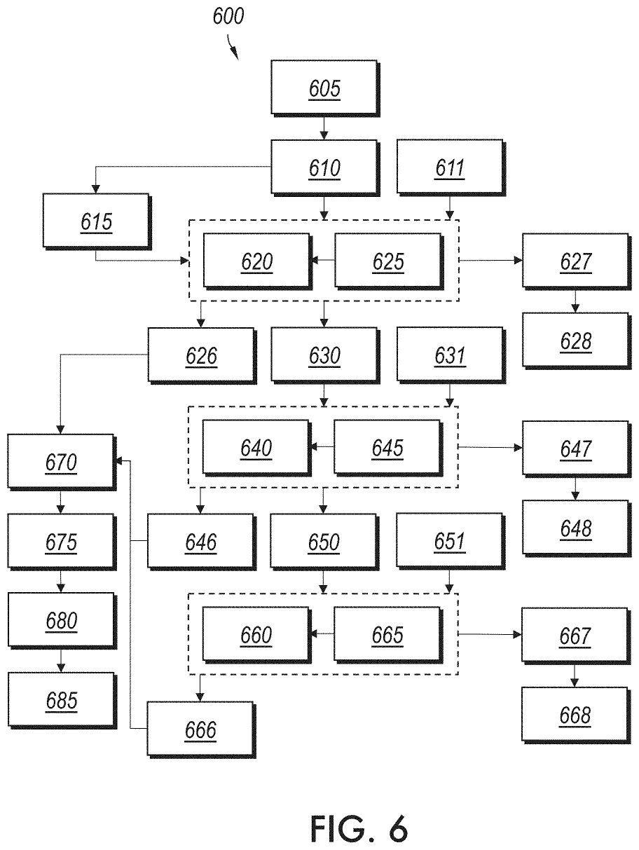

In some embodiments, the data may be fed to a system architecture, such as a Cygnus-X system architecture, to train a machine learning algorithm to identify a prescribed condition, such as right heart failure (RHF). In some embodiments, as shown in FIG. 4, the process 400 may include providing 410 a database, such as data from the national cardiogenic shock initiative (National CSI). The process may include matching 420 data from the database to pump data. The process also may include labelling 430 data to indicate whether such data was or was not associated with a target condition (such as RHF). The process may include identifying 440 key signals associated with the condition, then designing and training 450 the model as understood in the art, then validating 460 the model. In some embodiments, the process may include iterating the process, and may include enriching the process by, e.g., validating 470 using secondary datasets, such as from different databases or different studies, etc.

The models may be, e.g., neural networks. In some embodiments, the neural network may be a feed-forward neural network, a Radial Basis Function (RBF) Neural Network, a Multilayer Perceptron, a Convolutional Neural Network (CNN), or a Recurrent Neural Network (RNN).

In some embodiments, the system may be configured to receive cycles of gathering data, updating the probabilities, and gathering more data. In some cases, this may include sending and receiving information from a user-controlled device, and having the user perform one or more tasks to gather information.

A flowchart of this may be seen in FIG. 5. In some embodiments of a process 500, a hemodynamic support device may first be introduced 510 into a first area of a subject.

A first cycle 520 is then seen, where data may be received 521 from the hemodynamic support device, the processor(s) may determine 522 a probability of a condition existing and/or occurring. The processor(s) may then notify or display 523 the determined probability to the user, and optionally may display other data as disclosed herein.

As a result of this notification or display, the user (or an automated device) may then perform 550 a task, such as measuring a parameter of the subject, such as measuring a CVP.

A second cycle 540 is then seen, where data may be received 541, including the CVP value and optionally more data from the hemodynamic support device. The processor(s) may then determine 542 an updated probability of a condition existing and/or occurring based on the available data, including the CVP value, and may then notify or display 543 to the user the updated probability, and optionally other data as disclosed herein.

As a result of this second notification or display, the user (or an automated device) may then perform 570 another task, such as measuring a parameter of the subject, such as measuring PAP.

A third cycle 560 is then seen, where data may be received 561, including the PAP value and optionally more data from the hemodynamic support device. The processor(s) may, e.g., derive a PAPI value, and may determine 562 a further updated probability of a condition existing and/or occurring based on the available data, including the PAP and/or PAPI value, and may then notify or display 563 to the user the further updated probability, and optionally other data as disclosed herein.

As a result of this third notification, the user may, e.g., engage 570 in a treatment pathway. This may be a treatment pathway suggested by the one or more processors and displayed to the user.

It will be understood that different arrangements may be used. For example, in some embodiments, the user may decide to engage 570 in a treatment pathway after receiving the first or second notification. In some embodiments, multiple cycles without user actions being required may occur.

Thus, in some embodiments, the at least one processor may be configured to receive second data after receiving the data from the hemodynamic support device, where the second data relates to a third area of the subject's body that is different from the first area and the second area. For example, if the first area (where the hemodynamic support device is located) is the left heart, and second area (where the condition may exist) is the right heart, the third area may be, e.g.,, the vena cava or the pulmonary artery. Thus, the second data may include, e.g., a CVP or PAP value. In some embodiments, a medical instrument or device used to gather the second data also transmits the data to the at least one processor of the system. In other embodiments, the user may enter the data on their remote device (such as their phone, tablet, laptop, etc.), and causes the data to be transmitted to the at least one processor.

In some embodiments, the at least one trained ML algorithm may be further configured to determine a second probability of the condition based on the data from the hemodynamic support device and the second data.

Similarly, in some embodiments, the at least one processor may be configured to receive third data after receiving the second data from the user, the third data relating to a fourth area of the subject's body that is different from the first area, second area, and third area. In some embodiments, the at least one trained ML algorithm may be further configured to determine a third probability of the condition based on the data from hemodynamic support device, second data, and third data.

In some embodiments, the at least one processor may be further configured to derive at least one parameter from at least some of the received data, and the at least one trained ML algorithm is configured to determine a probability (such as the first, second, and/or third probability) further based on the at least one parameter. Non-limiting examples of such derived parameters include central venous pressure (CVP), a right atrial pressure (RAP), a minimum, maximum, and/or mean of RAP, right ventricle end-diastolic pressure (RVEDP), pulmonary artery pressure (PAP), a mean, systolic, and/or diastolic PAP, a pulmonary artery pressure index (PAPI), and/or an echo-based parameter of right heart function. In some embodiments, PAPI may be calculated by subtracting a diastolic pulmonary artery value (PAdia) from a systolic pulmonary artery value (PAsys), and the difference is then divided by RAP or CVP. In some embodiments, the echo-based parameter of right heart function is a right ventricle (RV) diameter, an RV volume, RV stroke volume index (RVSVI) value, RV stroke work index (RVSWI) value, and/or a tricuspid annular plane systolic excursion (TAPSE) value.

In some embodiments, the system may include a remote device, which may be configured to send the second data and third data to the at least one processor, and to receive the first probability, second probability, and third probability from the at least one processor. In some embodiments, no user-identifiable data is transmitted to or from the remote device.

Referring again to FIG. 1, in some embodiments, the system may include an additional device (or device) that includes a sensor. For example, in some embodiments the system may include a first device 150 (here, a patch) with a sensor 155. In some embodiments, each additional device may be applied in or on the subject in a different area of the body from other additional devices and from the first, second, third, and fourth areas of the body (here, the patch is applied to a chest of the subject, while the first through fourth areas were in and around the heart.

In some embodiments, the system may include a single additional device. In some embodiments, the system may include two or more additional devices. As shown in FIG. 1, a second additional device 151 (here, a watch or bracelet) is shown with sensor 156.

In some embodiments, the at least one trained ML algorithm may be configured to determine a probability (such as the first, second, or third probability) of the condition further based on data received from the sensor of the additional device.

The additional devices and associated sensors may provide data related to any appropriate parameters. For example, in some embodiments, the data received from the sensor of the additional device may include a heart rate, a value related to blood oxygen, a value relating to an electrocardiogram (ECG), a skin temperature, an acceleration, or a combination thereof.

In some embodiments, the at least one processor may be further configured to make determinations in an effort to aid in providing a human interpretation to the data. For example, in some embodiments, the at least one processor may be configured to determine if a probability (such as the first, second, and/or third probability) is above a first threshold and/or below a second threshold.

In some embodiments, the processor(s) may determine a trend over time in probabilities of the condition in the subject, and optionally may determine if a rate of change described by the trend is above a threshold rate and/or if the probability will be above the first threshold or below the second threshold within a predetermined period of time should the trend continue. For example, if a first threshold rate is 80%, a second threshold rate is 20%, and the most recent probability was 70%, that most recent probability determination would not be over the first threshold or under the second threshold, so in this example, it might not trigger an alarm. However, if the probability has been trending steadily upward from 45% to 70% in the past five minutes (e.g., the trend is defining a rate of change of +5% per minute), the probability could be estimated to exceed the 80% threshold in just over 2 minutes. If the predetermined period of time is 5 minutes (e.g., it is configured that something is not ideal if the system determines that high threshold may be exceeded within 5 minutes), then this determination may be useful.

In some embodiments, the processor(s) may be configured to identify one or more primary factors that result in the probability being above the first predetermined threshold and/or below the second predetermined threshold. For example, looking at a trained ML model, it may be evident what weights may be given to what features. For example, each such feature may be able to be assigned to different biological components, biological functions, and/or parameters (e.g., CVP, PAPI, etc.). With those elements, it may be relatively straightforward to determine what one or more components, functions, and/or parameters with the highest overall impact on a determined probability are. In some embodiments, a single component, function, and/or parameter may be identified. In some embodiments, multiple components, functions, and/or parameters may be identified.

In some embodiments, the processor(s) may determine a trend over time in identified primary factors. For example, the processor(s) may be configured to identify at least one factor every time a probability is determined to be over a threshold value. After 5 minutes, there may be a number of identified factors, which may be listed out on a display, for example, shown in a pareto chart. As will be appreciated other displays may be used in other embodiments. In some embodiments, the impact of multiple factors may be tracked over time, even if they are not considered a “primary” factor.

In some embodiments, some combination of these may be included.

In some embodiments, the at least one processor may be configured to generate an alert when the first probability is determined to be above the first predetermined threshold or below the second predetermined threshold, when the rate of change described by the trend is above the threshold rate, and/or when it is determined, should the trend continue, the probability will be above the first threshold or below the second threshold within the predetermined period of time. In some embodiments, the alert includes a visual signal to a user. In some embodiments, the alert may include an auditory or haptic signal to the user. In some embodiments, a text message or email is sent to a user. In some embodiments an alert may appear on a display on the controller. In some embodiments, an alert may appear on a user's remote device (e.g., laptop, mobile phone, etc.). In some embodiments, the alert may include one or more options for the user to select. In some embodiments, the alert may include text indicating tests and/or treatments for the condition.

In some embodiments, the at least one processor may be further configured to receive input from the user responsive to the alert. In some embodiments, this may include receiving data indicating a particular treatment was undertaken, or an existing treatment was stopped. In some embodiments, this may include receiving data indicating an option from the alert was selected.

In some embodiment, the at least one processor may be further configured to track the probability of the condition over time to determine if a provided treatment is effective at lowering risk of the condition. For example, if the input from the user responsive to the alert indicates the user has begun providing a specific treatment, the system may continue monitoring the probabilities over time to provide feedback to the user indicating whether the treatment is or has been effective at reducing or removing the condition.

In some embodiments, a method for detecting and/or inferring conditions may be provided. Referring to FIG. 6, the method 600 may include receiving 610 data from a hemodynamic support device that has been positioned in a first area of a subject's body. The method may include determining 620, with a trained machine learning algorithm, a first probability of a condition existing in a second area of the subject's body based on the received data, the second area being different form the first area.

As disclosed herein, in some embodiments, the method may include transmitting 615, over a network, the data from a first processor (that received the data from the hemodynamic support device) to a second processor, that is configured to use the trained machine learning algorithm to determine probabilities.

As disclosed herein, in some embodiments, the method may include transmitting 628, over a network, the first probability to at least one processor (such as the first processor, or a processor in a different remote device).

In some embodiments, the method may then include displaying 629 the first probability, or a text or image representative of the first probability. Other data, graphs, etc., as disclosed herein, may also be displaced. For example, this may be displayed on a console controlling the hemodynamic support device, on a mobile device, etc.

In some embodiments, the method may include providing 605 the at least one trained ML algorithms. In some embodiments, this may include providing a first ML algorithm trained on historical data gathered from a plurality of medical device product lines. In some embodiments, this may include providing a second ML algorithm trained on data gathered from a single medical device product line.

In some embodiments, the method may include receiving 630 second data, as disclosed herein, such as data from a user-controlled device, after receiving the data from the hemodynamic support device, the second data relating to a third area of the subject's body that is different from the first area and the second area. In some embodiments, the user may have entered the data into the device. In some embodiments, the device may have measured and sent the data.

In some embodiments, the method may include determining 640, using the at least one trained ML algorithm, a second probability of the condition based on the data from the hemodynamic support device and the second data.

As disclosed herein, in some embodiments, the method may include transmitting 648, over a network, the second probability to at least one processor (such as the first processor, or a processor in a different remote device).

In some embodiments, the method may then include displaying 649 the second probability, or a text or image representative of the second probability. Other data, graphs, etc., as disclosed herein, may also be displaced. This may be displayed, e.g., on a console controlling the hemodynamic support device, on a mobile device, etc.

In some embodiments, the method may include receiving 650 third data, as disclosed herein, such as from a user-controlled device, after receiving the second data from the user, the third data relating to a fourth area of the subject's body that is different from the first area, second area, and third area.

In some embodiments, the method may include determining 660, using the at least one trained ML algorithm, a third probability of the condition based on the data from hemodynamic support device, second data, and third data.

As disclosed herein, in some embodiments, the method may include transmitting 668, over a network, the third probability to at least one processor (such as the first processor, or a processor in a different remote device).

In some embodiments, the method may then include displaying 669 the third probability, or a text or image representative of the third probability. Other data, graphs, etc., as disclosed herein, may also be displaced. This may be displayed, e.g., on a console controlling the hemodynamic support device, on a mobile device, etc.

In some embodiments, prior to determining at least one probability, the method may include deriving 625, 645, 665 at least one parameter. As disclosed herein, the at least one parameter may be, e.g., central venous pressure (CVP), a right atrial pressure (RAP), a minimum, maximum, and/or mean of RAP, right ventricle end-diastolic pressure (RVEDP), pulmonary artery pressure (PAP), a mean, systolic, and/or diastolic PAP, a pulmonary artery pressure index (PAPI), and/or an echo-based parameter of right heart function.

In some embodiments, the method may include receiving data from an additional device (such as a wearable device or patch) including a sensor, as disclosed herein, where the sensor is positioned in or on a third area of the patient's body. As disclosed herein, in some embodiments, the at least one trained ML algorithm may determine the first probability further based on data received from the sensor of the additional device. As disclosed herein, the additional device may provide any of a wide range of relevant data. For example, in some embodiments, the sensor(s) in or on the additional device may collect data including a heart rate, a value related to blood oxygen, a value relating to an electrocardiogram (ECG), a skin temperature, an acceleration, or a combination thereof.

In some embodiments, the method may include making additional determinations 626, 646, 666 related to a determined probability. For example, in some embodiments, this may include, as disclosed herein, determining if a probability is above a first threshold and/or below a second threshold. In some embodiments, this may include determining a trend over time in probabilities of the condition in the subject, and optionally determining if a rate of change described by the trend is above a threshold rate and/or if the probability will be above the first threshold or below the second threshold within a predetermined period of time should the trend continue. In some embodiments, this may include identifying one or more primary factors that result in the probability being above the first predetermined threshold and/or below the second predetermined threshold. In some embodiments, this may include determining a trend over time in identified primary factors. In some embodiments, this may include a combination of these.

In some embodiments, the method may include alerting 670 a user when the first probability is determined to be above the first predetermined threshold or below the second predetermined threshold, when the rate of change described by the trend is above the threshold rate, and/or when it is determined, should the trend continue, the probability will be above the first threshold or below the second threshold within the predetermined period of time. In some embodiments, the alert may include, e.g., options for the user to select, the options including tests and/or treatments for the condition.

In some embodiments, the method may include receiving 675 input from the user responsive to the alert. In some embodiments, the input responsive to the alert may include data indicating a particular treatment was undertaken, or an existing treatment was stopped.

In some embodiments, the method may include tracking 680 the probability of the condition over time. In some embodiments, the method may include determining 685 (e.g., based on the tracked probabilities) if a provided treatment is effective at lowering risk of the condition.

Those skilled in the art will recognize or be able to ascertain using no more than routine experimentation, many equivalents to the specific embodiments of the present disclosure described herein. Such equivalents are intended to be encompassed by the following claims.

1. A system for detecting and/or inferring conditions, comprising:
a hemodynamic support device configured to be positioned in a first area of a subject's body;
at least one processor operably coupled to the hemodynamic support device, the at least one processor configured to:
receive data from the hemodynamic support device; and
determine, with at least one trained machine learning (ML) algorithm, a first probability of a condition existing in a second area of the subject's body based on the received data, the second area being different from the first area.

2. The system according to claim 1, wherein the at least one processor comprises:
a first processor configured to:
receive data from the hemodynamic support device; and
transmit the data to a second processor over a network; and
a second processor configured to:
receive data from the first processor; and
determine, with the trained machine learning algorithm, the first probability of the condition existing in the second area of the subject's body based on the received data.

3. The system according to claim 2, wherein the second processor is further configured to transmit the first probability to the first processor.

4. The system according to claim 2, further comprising a remote device,
wherein the second processor is further configured to transmit the first probability to the remote device; and
wherein the remote device is configured to display the first probability, or a text or image representative of the first probability.

5. The system according to claim 1, wherein the at least one trained ML algorithm comprises a first ML algorithm trained on historical data gathered from a plurality of medical device product lines.

6. The system according to claim 5, wherein the at least one trained ML algorithm further comprises a second ML algorithm trained on data gathered from a single medical device product line.

7. The system according to claim 6, wherein the at least one processor is configured to receive second data, after receiving the data from the hemodynamic support device, the second data relating to a third area of the subject's body that is different from the first area and the second area; and
wherein the at least one trained ML algorithm is further configured to determine a second probability of the condition based on the data from the hemodynamic support device and the second data.

8. The system according to claim 7, wherein the second data includes a value relating to a central venous pressure (CVP).

9. The system according to claim 7, wherein the at least one processor is configured to receive third data, after receiving the second data from the user, the third data relating to a fourth area of the subject's body that is different from the first area, second area, and third area; and
wherein the at least one trained ML algorithm is further configured to determine a third probability of the condition based on the data from hemodynamic support device, second data, and third data.

10. The system according to claim 9, wherein the third data includes a value relating to pulmonary artery pulsatility (PAP).

11. The system according to claim 9, wherein the at least one processor is further configured to derive at least one parameter, and the at least one trained ML algorithm is configured to determine the second probability and/or the third probability further based on the at least one parameter, where the at least one parameter is central venous pressure (CVP), a right atrial pressure (RAP), a min, max, and/or mean of RAP, right ventricle end-diastolic pressure (RVEDP), pulmonary artery pressure (PAP), a mean, systolic, and/or diastolic PAP, a pulmonary artery pressure index (PAPI), and/or an echo based parameter of right heart function.

12. The system according to claim 11, wherein PAPI is calculated by subtracting a diastolic pulmonary artery value (PAdia) from a systolic pulmonary artery value (PAsys), and the difference is then divided by RAP or CVP.

13. The system according to claim 11, wherein the echo-based parameter of right heart function is a right ventricle (RV) diameter, an RV volume, RV stroke volume index (RVSVI) value, RV stroke work index (RVSWI) value, and/or a tricuspid annular plane systolic excursion (TAPSE) value.

14. The system according to claim 9, further comprising a remote device, the remote device configured to send the second data and third data to the at least one processor, and to receive the first probability, second probability, and third probability from the at least one processor.

15. The system according to claim 14, where no user-identifiable data is transmitted to or from the remote device.

16. (canceled)

17. The system according to claim 1, further comprising an additional device operably coupled to the at least one processor, the additional device including a sensor, the sensor being positioned in or on a third area of the patient's body.

18-19. (canceled)

20. The system according to claim 17, wherein the at least one trained ML algorithm is configured to determine the first probability further based on data received from the sensor of the additional device.

21. The system according to claim 20, wherein the data received from the sensor of the additional device includes a heart rate, a value related to blood oxygen, a value relating to an electrocardiogram (ECG), a skin temperature, or an acceleration.

22. The system according to claim 1, wherein the first area of the patient's body is the left heart, and the second area of the patient's body is the right heart.

23. The system according to claim 22, wherein the data from the hemodynamic support device includes first information related to left heart contractile function, and second information related to suction or pump flow in the left heart.

24. The system according to claim 23, wherein the first information includes left ventricle (LV) contractility, aortic (AO) pulse pressure and/or pulsatility, or a combination thereof.

25. The system according to claim 1, wherein the data from the hemodynamic support device includes aortic (AO) pressure, left ventricular (LV) pressure, pump motor speed, pump motor current, LV-AO pressure gradient, pump flow, cardiac output, native cardiac output, LV pulse rate, AO pulse rate, or a combination thereof.

26. The system according to claim 25, wherein the data from the hemodynamic support device also includes LV volume via conductance, heart rate, heart rhythm, arterial pressure, blood oxygenation, or a combination thereof.

27. The system according to claim 26, wherein the at least one processor is further configured to derive at least one parameter, and the at least one trained ML algorithm is configured to determine the first probability based on the data from the hemodynamic support device and the at least one parameter, where the at least one parameter is: a LV end-diastolic pressure (LVEDP); a pump suction; a pump alarm rate and/or type; a cardiac power output; a LV contractility; a LV relaxation; a pulse wave velocity; an ejection fraction; a statistical metric of a parameter included in the data from the hemodynamic support device; and/or a systolic value, diastolic value, mean, median, min, max, delta, or pulse of a parameter included in the data from the hemodynamic support device.

28. The system according to claim 1, wherein the at least one processor is further configured to:
determine if the first probability is above a first threshold and/or below a second threshold;
determine a trend over time in probabilities of the condition in the subject, and optionally if a rate of change described by the trend is above a threshold rate and/or if the probability will be above the first threshold or below the second threshold within a predetermined period of time should the trend continue;
identify one or more primary factors that result in the probability being above the first predetermined threshold and/or below the second predetermined threshold;
determine a trend over time in identified primary factors; or
a combination thereof.

29. The system according to claim 28, wherein the at least one processor is further configured to alert a user when the first probability is determined to be above the first predetermined threshold or below the second predetermined threshold, when the rate of change described by the trend is above the threshold rate, and/or when it is determined, should the trend continue, the probability will be above the first threshold or below the second threshold within the predetermined period of time.

30. The system according to claim 29, wherein the at least one processor is further configured to receive input from the user responsive to the alert.

31-32. (canceled)

33. The system according to claim 1, wherein the at least one processor is further configured to track the probability of the condition over time to determine if a provided treatment is effective at lowering risk of the condition.

34. A method for detecting and/or inferring conditions, comprising:
receiving data from a hemodynamic support device positioned in a first area of a subject's body; and
determining, with a trained machine learning algorithm, a first probability of a condition existing in a second area of the subject's body based on the received data, the second area being different form the first area.

35-69. (canceled)
